(19) United States
(12) Patent Application Publication
(10) Pub. No.: US 2026/0274812 A1
(43) Pub. Date: Sep. 17, 2026

(54) PRODUCING 2,5-FURANDICARBOXYLIC ACID FROM MIXED FEED

(71) Applicant: Furanix Technologies B.V., Amsterdam (NL)

(72) Inventors: Jeffrey John Kolstad, Amsterdam (NL); Jan Hendrik Blank, Amsterdam (NL); Gerardus Johannes Maria Gruter, Amsterdam (NL)

(21) Appl. No.: 18/871,556

(22) PCT Filed: Jun. 13, 2023
(86) PCT No.: PCT/EP2023/065712

§ 371 Date: Dec. 4, 2024

(30) Foreign Application Priority Data
Jun. 14, 2022 (EP) 22178819.3

(51) Int. Cl. C07D 307/68

(52) U.S. Cl. CPC C07D 307/68; C07B 2200/13

(57) ABSTRACT

Process for producing 2,5-furandicarboxylic acid, comprising the steps of: a) oxidizing feed comprising 5-methylfurfural using an oxidizing gas at a temperature in the range of 150 to 210° C. to obtain a crude carboxylic acid composition comprising 2,5-furandicarboxylic acid in the presence of acetic acid and a catalyst system comprising cobalt, manganese and bromine; and b) separating at least a portion of the solid 2,5-furandicarboxylic acid from the crude carboxylic acid composition to obtain crude solid 2,5-furandicarboxylic acid, wherein the feed further comprises alkoxymethyl-2,5-furfural of which the alkoxy group comprises of from 1 to 3 carbon atoms.

### TECHNICAL FIELD

The present invention relates to a process for producing 2,5-furandicarboxylic acid, specifically a process for producing 2,5-furandicarboxylic acid using 5-methylfurfural as starting material.

### BACKGROUND

2,5-Furandicarboxylic acid (FDCA) is known in the art to be a highly promising building block for replacing petroleum-based monomers in the production of high performance polymers. In recent years, 2,5-furandicarboxylic acid and the novel plant-based polyester polyethylenefuranoate (PEF), a completely recyclable plastic with superior performance properties compared to today's widely used petroleum-based plastics, have attracted a lot of attention. These materials could provide a significant contribution to reducing the dependence on petroleum-based polymers and plastics, while at the same time allowing for a more sustainable management of global resources. Comprehensive research was conducted in the field to arrive at a technology for producing 2,5-furandicarboxylic acid and PEF in a commercially viable way.

2,5-Furandicarboxylic acid is typically obtained by oxidation of molecules having furan moieties, e.g. 5-hydroxymethylfurfural (5-HMF) as well as the corresponding esters and ethers, e.g. 5-alkoxymethylfurfural, and similar starting materials, that are typically obtained from plant-based sugars, e.g. by sugar dehydration. A broad variety of oxidation processes is known from the prior art such as enzymatic and metal catalysed processes, either heterogeneous or homogeneous. WO 2014/014981, WO2012/161968 and WO 2011/043661 describe processes using catalyst systems comprising cobalt, manganese and bromine to oxidize compounds having a furan moiety to 2,5-furandicarboxylic acid using oxygen or air as an oxidizing agent.

The purity of crude 2,5-furandicarboxylic acid product is oftentimes not sufficient for use in the manufacture of polymers having desirable properties. The product obtained by oxidation can be colored. Color is disadvantageous in that it indicates the presence of impurities. In order to objectively determine color, absorbance of a solution is to be measured at a particular wavelength such as 400 nm. Impurities which lead to color are often difficult to specifically identify making their removal difficult.

Processes have been developed for further purifying crude oxidation products. Exemplary purification processes are disclosed in WO 2014/014981 and WO 2016/195499.

It can be advantageous to be able to use 5-methylfurfural (5-MF) for oxidation into 2,5-furandicarboxylic acid as the availability of 5-methylfurfural may increase in the future. Processes which produce 5-hydroxymethylfurfural (5-HMF), as well as corresponding esters or ethers, frequently use sugar as feedstock such as glucose or especially fructose. In contrast, processes to make 5-methylfurfural can start from biomass or cellulosic materials which do not compete with the food chain and which may have reduced environmental impact. Furthermore, it could be expected that less by-products are formed in the conversion of 5-methylfurfural due to methyl group which tends to be less reactive.

While oxidation of compounds such as 5-hydroxymethylfurfural and ethers thereof has been extensively studied, less is known about oxidation of 5-methylfurfural. Soviet Union Inventor's Certificate 441877 describes the conversion of 5-methylfurfural into 2,5-furandicarboxylic acid at low yield and unknown purity. WO2011043661 mentions 5-methylfurfural as a possible feed for oxidation. Examples 3a and 3b using 5-methylfurfural gave a lower yield of 2,5-furandicarboxylic acid than Examples 1d and 1 h obtained from 5-hydroxymethylfurfural, both at 100% conversion. Therefore, the product obtained from 5-methylfurfural contained more compounds other than the free diacid, i.e. 2,5-furandicarboxylic acid, than the product obtained from 5-hydroxymethylfurfural.

U.S. Pat. No. 9,321,744 describes preparing 2,5-furandicarboxylic acid by oxidizing 5-alkoxymethyl-2-furoic acid wherein the alkoxy group contains of from 1 to 9 carbon atoms in combination with a second furan compound. The second furan compound can be chosen from a wide group of compounds. Example 3 used a feed comprising 3 g of 5-methylfurfural and 1.5 g of 5-(acetoxymethyl)-2-furoic acid of which 99.29% was converted in the oxidation. The 5-(acetoxymethyl)-2-furoic acid was prepared by reacting 2,5-hydroxymethylfurfural with triethylamine and acetic anhydride for 14 hours, acidifying, extracting the acidified solution, drying the organic phase, washing the solid obtained and finally again drying. It is disadvantageous if a process requires feed which is cumbersome to prepare.

In addition, it was also found that crude 2,5-furandicarboxylic acid obtained from 5-methylfurfural can suffer from incorporation of catalyst metals, more specifically manganese and/or cobalt, into the product cake. This not only contaminates the product but also withdraws valuable catalyst components from the system that could otherwise be reused or recycled.

### DISCLOSURE OF THE INVENTION

It was an objective to improve the purity of the crude carboxylic acid obtained by oxidation of 5-methylfurfural. A further objective was to improve, i.e. reduce, the absorbance of the crude carboxylic acid obtained. Furthermore, there was a desire for a process for producing 2,5-furandicarboxylic acid from 5-methylfurfural that reduces the problem of incorporation of metals into the crude solid 2,5-furandicarboxylic acid preferably combined with increased retention of the catalyst metals in the liquid separated from the solid 2,5-furandicarboxylic acid.

Surprisingly, it now has been found that the absorbance of solid 2,5-furandicarboxylic acid can be improved by using a feed comprising alkoxymethyl-2,5-furfural besides 5-methylfurfural. An additional advantage is that it was found that the process allowed to obtain desirable products in high yield.

The invention relates to a process for producing 2,5-furandicarboxylic acid comprising the steps of: a) oxidizing feed comprising 5-methylfurfural using an oxidizing gas at a temperature in the range of 150 to 210° C. to obtain a crude carboxylic acid composition comprising 2,5-furandicarboxylic acid in the presence of acetic acid and a catalyst system comprising cobalt, manganese and bromine; and b) separating solid 2,5-furandicarboxylic acid from the crude carboxylic acid composition to obtain crude solid 2,5-furandicarboxylic acid, wherein the feed further comprises alkoxymethyl-2,5-furfural of which the alkoxy group comprises of from 1 to 3 carbon atoms.

### MODES FOR CARRYING OUT THE INVENTION

The process is aimed at producing 2,5-furandicarboxylic acid. The final product obtained can contain further compounds besides 2,5-furandicarboxylic acid especially if the final product obtained is the crude solid 2,5-furandicarboxylic acid produced in step b). The product obtained after the washing of step c) or the hydrogenation steps d)-f), which steps are discussed later, tends to contain a lower amount of further compounds besides 2,5-furandicarboxylic acid.

Besides the feed, the mixture present in step a) comprises oxidizing gas, acetic acid and a catalyst system. After the reaction has started, the mixture present in step a) tends to further contain water produced by oxidation of 5-methylfurfural.

The feed for use in the present process comprises alkoxymethyl-2,5-furfural besides 5-methylfurfural. The alkoxy group of the alkoxymethyl-2,5-furfural contains of from 1 to 3 carbon atoms. The alkoxy group can for example be ethoxy. Most preferably, the alkoxymethyl-2,5-furfural is methoxymethylfurfural.

The feed can comprise further compounds besides 5-methylfurfural and alkoxymethyl-2,5-furfural. Generally, such compounds would be present in a limited amount only. Preferably, the feed consists for at least 80% wt of 5-methylfurfural and alkoxymethyl-2,5-furfural of which the alkoxy group comprises of from 1 to 3 carbon atoms and preferably is methylmethoxy-2,5-furfural, more preferably for at least 90% wt, most preferably for at least 95% wt. Most preferably, the feed consists of 5-methylfurfural and alkoxymethyl-2,5-furfural of which the alkoxy group comprises of from 1 to 3 carbon atoms.

It is preferred for the feed to comprise 5-methylfurfural and alkoxymethyl-2,5-furfural in a weight ratio of 5-methylfurfural to alkoxymethyl-2,5-furfural of from 95:5 to 30:70, more specifically of from 90:10 to 50:50 based on total amount of feed.

It is possible to additionally add in step a) a modifying acid to further improve the purity of the crude solid 2,5-furandicarboxylic acid and thereby its properties. Such modifying agent can be selected from the group consisting of carboxylic acids having a pKa of less than 3.2, more especially mono- and dicarboxylic acids having from 2 to 5 carbon atoms. More preferably, the modifying acid is selected from the group consisting of bromoacetic acid, dibromoacetic acid, 5-bromo-2-furoic acid, fumaric acid, acetoxy-acetic acid, maleic acid and furoic acid, and more preferably is selected from the group consisting of bromoacetic acid, dibromoacetic acid, acetoxy-acetic acid and 5-bromo-2-furoic acid. Preferably, the amount of modifying acid added is of from 0.5 to 6% by weight based on weight amount of acetic acid present in step a), preferably of from 1 to 5% by weight. Alternatively, modifying acid can be obtained from the crude solid 2,5-furandicarboxylic acid by separating 5-bromo-2-furoic acid. Therefore, a preferred embodiment comprises (i) obtaining at least part of the 5-bromo-2-furoic acid from the crude solid 2,5-furandicarboxylic acid, and (ii) adding at least part of the 5-bromo-2-furoic acid obtained as modifying acid to step a). The 5-bromo-2-furoic acid can be obtained in any way known to the person skilled in the art. Preferably, 5-bromo-2-furoic acid is obtained from the crude solid 2,5-furandicarboxylic acid by washing with or reslurrying the crude solid 2,5-furandicarboxylic acid in acetic acid and/or water to obtain a solution containing 5-bromo-2-furoic acid.

Step a) preferably is carried out at a temperature in the range of 150 to 210° C., preferably a temperature of 160 to 190° C., more preferably a temperature in the range of from 165 to 180° C. . . . Preferably, the pressure in step a) is in the range of 700 to 2000 kPa. These parameters were found to produce 2,5-furandicarboxylic acids of good purity in good yields while at the same time enabling the reactors to be run such that the substantial heat generated by oxidation is removed by vaporization of a portion of the solvent. This is known in the art as adiabatic operation.

The catalyst system comprises cobalt, manganese and bromine either as the element or as a derivative thereof. The catalyst system preferably has a weight ratio of cobalt to manganese in the catalyst system of 10 or higher, preferably 15 or higher, and/or a weight ratio of bromine to the combined weight of cobalt and manganese in the catalyst system of 1 or higher, preferably 1.5 or higher, most preferably 2 or higher, wherein the value is preferably less than 4.0, more preferably less than 3.5. If the catalyst system comprises other metals besides cobalt and manganese in an amount of 5% by weight or more, it is preferred that the above ratios are achieved for the weight ratio of bromine to the combined weight of all metals in the catalyst system. The metals preferably are added as salts which are soluble in the reaction mixture. Typically, the amount of cobalt is selected in the range of 500 to 6000 ppm by weight, based on the weight of the feed, acetic acid and catalyst system. The amount of manganese typically is in the range from 20 to 6000 ppm by weight, based on the weight of the feed, acetic acid and catalyst system Typically, the bromine concentration would be from 30 to 8000, preferably 50 to 4500 ppm by weight of bromine, based on weight of the feed, acetic acid and catalyst system. Alternatively, the bromine content is from 3000 to 8000 ppm by weight.

The oxidizing gas can be any gas known to be suitable by the person skilled in the art. Preferably, the oxidizing gas comprises molecular oxygen. Most preferably, the oxidizing gas is air.

The reactor for carrying out the oxidation can be any typical oxidation reactor that is known in the art.

A post-oxidation step has been found to be preferred especially when employed at high temperature. Most preferred is a process wherein a post oxidation step a1) is applied after step a) at a temperature of at a temperature in the range of 150 to 210° C., more specifically of 160 to 210° C.

In step b) solid 2,5-furandicarboxylic acid is separated. This means that solid containing 2,5-furandicarboxylic acid is separated from the crude carboxylic acid composition. Not all of the 2,5-furandicarboxylic acid generally will be removed from the crude carboxylic acid composition while generally not all of the solid which is separated will be 2,5-furandicarboxylic acid.

Preferably, at least 50% by weight with respect to the weight of the dry crude solid 2,5-furandicarboxylic acid will be 2,5-furandicarboxylic acid, more preferably at least 70% by weight, more preferably at least 80% by weight, more preferably at least 90% by weight, most preferably at least 95% by weight. Other compounds which can be present as part of the crude solid 2,5-furandicarboxylic acid are derivatives of 2,5-furandicarboxylic acid such as methyl ester of 2,5-furandicarboxylic acid, 5-hydroxymethyl-furan-2-carboxylic acid (HMFCA), 2-carboxy-5-(formyl) furan (FFCA), 5-bromo-2-furoic acid (Br-FCA) and bis-carbonyl-furoic acid (BCFCA).

In step b) solid 2,5-furandicarboxylic acid is separated. This means that solid containing 2,5-furandicarboxylic acid is separated from the crude carboxylic acid composition. Not all of the 2,5-furandicarboxylic acid generally will be removed from the crude carboxylic acid composition while generally not all of the solid which is removed will be 2,5-furandicarboxylic acid.

Preferably, at least 50% by weight with respect to the weight of the dry crude solid 2,5-furandicarboxylic acid will be 2,5-furandicarboxylic acid, more preferably at least 70% by weight, more preferably at least 80% by weight, more preferably at least 90% by weight, more preferably at least 95% by weight, more preferably at least 98% by weight. Other compounds which can be present as part of the crude solid 2,5-furandicarboxylic acid are ethers and esters of 2,5-furandicarboxylic acid such as methyl ester of 2,5-furandicarboxylic acid.

In step b) at least a portion of the solid 2,5-furandicarboxylic acid is separated, that means separated from the crude carboxylic acid composition. The separation can be carried out in any way known to the person skilled in the art such as a solid-liquid separation zone in which a solid cake and a mother liquor are generated.

In a solid-liquid separation zone, a solid crude 2,5-furandicarboxylic acid cake and a mother liquor tend to be generated by separating the solid 2,5-furandicarboxylic acid. In continuous operation, at least a portion, preferably at least 60% by weight, more preferably at least 80% by weight, of the mother liquor preferably is routed from the solid-liquid separation zone to the reactor in which the oxidation occurs, also referred to as oxidation reactor, as recycled mother liquor stream.

The process of the present invention provides good results for batch processes, wherein e.g. solid precipitate comprising crude carboxylic acid composition is taken from the batch reactor, and processed in a separation zone according to step b). The different components of the feed can be added to the oxidation reactor separately. It is possible to complete a first batch process in order to analyze the resulting crude carboxylic acid composition and to change the feed composition in a subsequent batch run in case the absorbance and/or metal in the crude carboxylic acid composition of the first run exceeds the desired amounts.

The process of the present invention shows its full potential in continuous or semi-continuous processes as these processes are in need for suitable controlling mechanisms that allow for a minimal invasive adjustment of the running system that is suitable to counter the problem of absorbance and/or metal incorporation into the crude solid 2,5-furandicarboxylic acid. Such processes generally involve continuous or intermittent addition of oxidizable feed and withdrawal of crude carboxylic acid composition comprising 2,5-furandicarboxylic acid.

Preferred is a process according to the invention, wherein the crude solid 2,5-furandicarboxylic acid comprises 2,5-furandicarboxylic acid in an amount greater than 95%, preferably greater than 98%, by weight with respect to the weight of the dry solids.

Preferably, the crude solid 2,5-furandicarboxylic acid comprises a combined amount of cobalt and manganese of less than 3000 parts per million by weight (ppm), preferably less than 2000 ppm, preferably less than 1000 ppm, more preferably less than 700 ppm by weight of metal with respect to the weight of the 2,5-furandicarboxylic acid in the crude solid 2,5-furandicarboxylic acid.

In a preferred embodiment, the process further comprises d) contacting washed crude solid obtained in step c) with polar solvent to obtain a solution; e) contacting the solution with hydrogen in the presence of a hydrogenation catalyst at hydrogenation conditions yielding a hydrogenated solution; and f) separating purified 2,5-furandicarboxylic acid from the hydrogenated solution, preferably separating by crystallization. Suitable process conditions are for example described in WO2016/195490. Preferred process conditions comprise contacting with hydrogen at a temperature in the range of 150 to 200° C. and a contact time with the hydrogenation catalyst in the range of 5 seconds to 15 min.

Step d) suitably comprises mixing the sold obtained in step c) with polar solvent to substantially fully dissolve the 2,5-furandicarboxylic acid and any further furan containing compounds. Preferably, the polar solvent is selected from the group consisting of water, acetic acid and mixtures thereof.

It will be clear to the person skilled in the art that preferably all solution is subjected to step e) although it is possible to use part of the solution only.

Hereinafter, the invention is described in more detail using experiments.

### EXAMPLES

The oxidation reactor is a 600 ml stirred pressure vessel, with two impellors. The reactor is pre-charged with a mixture having a total weight of 310 grams. The mixture comprises catalyst components provided as cobalt (II) acetate tetrahydrate, manganese (II) acetate tetrahydrate, and HBr as 48% by weight (wt %) in water. The amounts of the catalyst components are such as to yield a mixture which contained 3300 ppm Co, 188 ppm Mn and 7000 ppm Br. Water is added in an amount to result in 5 wt % of the total mixture, after accounting for the water introduced as part of the catalyst components. The balance is acetic acid.

The oxidation reactor is purged, pressurized, and heated to the desired operating temperature with stirring at 2000 rpm. The feed of Comparative Experiment 1 was 5-methoxymethylfurfural (MMF), the feed for Comparative Experiments 2 to 4 was 5-methyl furfural (5-MF) and the feed for Experiment 5 (according to the invention) was a mixture of 5-methyl furfural (MF) and 5-methylmethoxy furfural (MMF) (weight ratio 70/30).

The process is started with a typical feed rate 8.3 mmol/minute. This feed rate was continued for 60 minutes (total feed 500 mmol) in the first set of experiments (Comp. 1, Comp. 2 and Comp. 3) and for 30 minutes (total feed 250 mmol) in the second set of experiments (Comp. 4 and Example 5).

The oxidation reactor was purged, pressurized and heated to the desired operating temperature with stirring at 2000 rpm. A flow rate of lean air (8% oxygen) is started at a typical flow rate of 10 normal L/minute. The reaction typically begins within 3 minutes, noticed by a sharp decrease in oxygen in the outlet and an increase in CO and CO₂. During the reaction heat is generated, and a vapor stream is taken overhead and condensed. This vapor stream comprises mainly of acetic acid and water. The amount of solvent captured in the overhead is continuously monitored, and made up in the oxidation reactor with a fresh flow of solvent to the reactor.

The typical operating pressure was 12 to 14 barg at 160° C. oxidation temperature.

At the end of the desired feed period, the feed of oxidizable compound is stopped, and the contents of the oxidation reaction is subjected to a period of post-oxidation.

The oxidation was followed by post-oxidation. Post-oxidation was conducted by stopping the flow of lean air for 1 minute and then reestablishing lean air flow at 4 Nl/min for 20 minutes while maintaining the reaction temperature at 160° C.

Solids were separated by filtration and the cake obtained was washed with 1 part solvent (95 acetic acid to 5 parts water, by weight) to 1 part estimated dry cake weight.

The yield is the molar ratio of FDCA and monoester of FDCA both as present in the washed crude solid 2,5-furandicarboxylic acid with respect to the total molar amount of furanic compounds in the feed i.e. MF and MMF. Partially oxidized compounds such as 5-formyl-2-furoic acid are not considered desired product and are disregarded for the yield.

The color of the cake was assessed visually.

The cake absorbance was measured by mixing 300 mg of crude 2,5-furandicarboxylic acid with 10 ml of dimethyl sulfoxide (DMSO). To ensure complete dissolution, the solution was allowed to stand for 4 hours. The absorbance of this solution was measured in a 1 cm cell in a UV/VIS photo-spectrometer against a DMSO standard using a wavelength of 400 nm.

The amount of cobalt and manganese were determined by inductively coupled plasma or ICP analysis.

The retention of manganese in the mother liquor is the amount of manganese in the mother liquor (weight of mother liquor times concentration of manganese) with respect to the total amount of manganese in the initial pre-charge.

The composition of the feed of Example 5 is the weight ratio of 5-methylfurfural and alkoxymethyl-2,5-furfural, respectively, to total amount of feed.

The results are shown in the below table.

TABLE 1
Oxidation of feed

| | | | | | | Mn | |
|---|---|---|---|---|---|---|---|
| | | Amount | UV | | | retention | |
| | | of feed | absorbance | Co | Mn | in mother | Yield |
| # | Feed | (mmol) | 400 nm | (ppm) | (ppm) | liquor | (% wt) |
| Comp. 1 | MMF | 500 | 0.020 | 2240 | 460 | 4% | 87.6 |
| Comp. 2 | MF | 500 | 0.110 | 2120 | 360 | 15% | 70.2 |
| Comp. 3 | MF | 500 | 0.259 | 2490 | 400 | 17% | 68.8 |
| Comp. 4 | MF | 250 | 0.053 | 1970 | 430 | 25% | 70.4 |
| Example | 70 MF/ | 250 | 0.014 | 425 | 176 | 61% | 75.4 |
| 5 | 30 MMF | | | | | | |

Table 1 shows that a feed comprising a mixture of 5-methylfurfural and alkoxymethyl-2,5-furfural resulted in solid 2,5-furandicarboxylic acid having improved absorbance at 400 nm compared with 5-methylfurfural and alkoxymethyl-2,5-furfural per se. Additionally, the solid 2,5-furandicarboxylic acid obtained from a mixture of 5-methylfurfural and alkoxymethyl-2,5-furfural contained a reduced amount of both cobalt and manganese compared with solid 2,5-furandicarboxylic acid obtained from 5-methylfurfural and alkoxymethyl-2,5-furfural per se while the mother liquor contained a higher amount of manganese. Furthermore, a high yield of desirable products was obtained with a mixture of 5-methylfurfural and alkoxymethyl-2,5-furfural.

1. A process for producing 2,5-furandicarboxylic acid, comprising the steps of:
a) oxidizing feed comprising 5-methylfurfural using an oxidizing gas at a temperature in the range of 150 to 210° C. to obtain a crude carboxylic acid composition comprising 2,5-furandicarboxylic acid in the presence of acetic acid and a catalyst system comprising cobalt, manganese and bromine; and
b) separating solid 2,5-furandicarboxylic acid from the crude carboxylic acid composition to obtain crude solid 2,5-furandicarboxylic acid,
wherein the feed further comprises alkoxymethyl-2,5-furfural of which the alkoxy group comprises of from 1 to 3 carbon atoms.

2. The process according to claim 1, wherein the alkoxymethyl-2,5-furfural is methoxymethylfurfural.

3. The process according to claim 1, wherein the feed comprises 5-methylfurfural and alkoxymethyl-2,5-furfural in a weight ratio of 5-methylfurfural to alkoxymethyl-2,5-furfural of from 90:10 to 50:50.

4. The process according to claim 1, wherein the feed consists of alkoxymethyl-2,5-furfural and 5-methylfurfural.

5. The process according to claim 1 wherein the step a) further comprises modifying acid selected from the group consisting of mono- and dicarboxylic acids having from 2 to 5 carbon atoms, preferably selected from the group consisting of monocarboxylic acids having from 2 to 5 carbon atoms and more preferably selected from the group consisting of bromoacetic acid, dibromoacetic acid, acetoxy-acetic acid and 5-bromo-2-furoic acid.

6. The process according to claim 5, wherein the amount of modifying acid is of from 1 to 5% by weight based on weight amount of solvent acid present in step a).

7. The process according to claim 1, wherein the process further comprises
c) washing the crude solid 2,5-furandicarboxylic acid with a first washing solution comprising a saturated organic acid solvent having from 2 to 6 carbon atoms and less than 15% by weight of water, based on total amount of washing solution, preferably followed by washing with a second washing solution comprising water in an amount of more than 95% by weight with respect to the weight of the washing solution.

8. The process according to claim 1, wherein the crude solid 2,5-furandicarboxylic acid comprises a combined amount of cobalt and manganese of less than 1000 ppm, preferably less than 700 ppm, by weight as metal with respect to the weight of the 2,5-furandicarboxylic acid in the crude solid 2,5-furandicarboxylic acid.

9. The process according to claim 1, wherein step b) comprises
separating solid 2,5-furandicarboxylic acid from the crude carboxylic acid composition in a solid-liquid separation zone to obtain crude solid 2,5-furandicarboxylic acid and a mother liquor, at least part of which mother liquor is recycled to step a).

10. The process according to claim 7, which process further comprises
d) contacting washed crude solid obtained in step c) with polar solvent to obtain a solution;
e) contacting the solution with hydrogen in the presence of a hydrogenation catalyst at hydrogenation conditions yielding a hydrogenated solution; and
f) separating purified 2,5-furandicarboxylic acid from the hydrogenated solution, preferably separating by crystallization.
